(19) United States
(12) Patent Application Publication
(10) Pub. No.: US 2003/0152239 A1
(43) Pub. Date: Aug. 14, 2003

(54) Resonator

(75) Inventor: Andreas Graefenstein, Karlsruhe (DE)

Correspondence Address: CROWELL & MORING LLP, INTELLECTUAL PROPERTY GROUP, P.O. BOX 14300, WASHINGTON, DC, 20044-4300, US

(73) Assignee: Filterwerk Mann & Hummel GmbH, Ludwigsburg (DE)

(21) Appl. No.: 10/345,414

(22) Filed: Jan. 16, 2003

(30) Foreign Application Priority Data
Jan. 17, 2002 (DE) 102 01 494.9

(51) Int. Cl. H04R 25/00; F01N 1/02; H04R 1/02; A61F 11/06; G10K 11/16; H03B 29/00; H04R 1/20

(52) U.S. Cl. 381/71.5; 381/353; 381/190; 181/250; 381/71.7

(57) ABSTRACT

A resonator for noise damping in a sound-conducting tubular channel (2) is provided with an actively controllable sound generator (4) which is arranged in a resonator chamber (12) to generate interference noise (16) to be superimposed on the tubular channel noise. The resonator chamber (12) is connected via a sound-transmitting opening in a tube wall (3) of the tubular channel (2) to the inside of the tubular channel. A measurement signal from a sound sensor (8) arranged in the tubular channel (2), containing information about the sound spectrum in the channel, is connected amplified, to the sound generator (4) with the same frequency and with inverse phase position. To amplify the active suppression of the sound level by the resonator with a resonator of the smallest possible overall size, the sound-transmitting opening in the tube wall is constructed as a peripheral annular gap in the tube wall.

### BACKGROUND OF THE INVENTION

The present invention relates to a resonator for noise damping in a sound-conducting tubular channel.

To reduce the noise emission of air-conducting tubular channels in motor vehicles, for example, active and passive measures are known. Absorption dampers such as damping materials and the like or resonance dampers are used for passive sound level damping. Furthermore, shunt and/or series resonators and the quarter-wave tube are known as passive components, which are associated with the sound-conducting tubular channel to be damped, and which are designed for the frequency range to be damped through suitable geometric layout. However, a large structural volume is required for this purpose, which may be unavailable, particularly in the construction of engines for motor vehicles. Furthermore, changes of the spatial properties for the installation of typical passive resonators usually lead to costly construction changes.

An active resonator for damping noise production in an exhaust line of an internal combustion engine is known from WO 93/25999. The known resonator comprises a loudspeaker as an actively controllable sound generator, which is positioned in a resonator chamber, constructed inside a resonating body, to generate an anti-phase noise to be superimposed on the tubular channel sound. A connecting sleeve is constructed in the tube wall of the sound-conducting tubular channel, to which the can-shaped resonating body is attached and which forms a sound-transmitting opening between the resonator chamber and the tubular channel. The loudspeaker is driven by a control unit to generate a sound signal, which, through modification in the resonator chamber, is superimposed as anti-phase noise upon the sound to be damped in the tubular channel and thus produces sound damping. The control unit uses a measurement signal from a sound sensor in the tubular channel, i.e. a feedback signal, which contains information about the remaining sound level after the damping, for regulating the noise.

German Patent No. 198 61 018 C2 discloses a controlled acoustic waveguide for sound damping, in which an oblong hollow resonator chamber is connected through a sound-transmitting opening to the sound-conducting tubular channel. The lengthwise resonances of the resonator chamber may be actively influenced using a sound generator which is positioned on the face of the resonator chamber lying opposite the opening to the sound-conducting tubular channel. To determine the noise signal of the sound generator, a sensor for detecting the sound spectrum in the tubular channel is positioned in the tubular channel and a microphone, which detects the membrane oscillations of the sound generator, is provided directly in front of the membrane of the sound generator. The microphone signal is inverted using an amplifier and fed back amplified to the loudspeaker as a function of the sound signal of the sensor in the tubular channel.

The known active resonance resonators require a large structural volume, which is not always available, for example, in automotive engineering. Particularly for damping lower frequencies, below 100 Hz, the known devices require larger resonance body volumes and large geometric dimensions as a function of the damping to be achieved. If only a small overall space is available for devices for noise level damping, the use of known resonators having large dimensions is often excluded or their use is only possible in a limited way.

### SUMMARY OF THE INVENTION

It is an object of the present invention to provide a noise damping resonator which makes possible a strong active suppression of the sound level.

Another object of the invention is to provide an effective noise damping resonator which has a low overall resonator space requirement, i.e., a low resonator volume.

These and other objects are achieved in accordance with the invention by providing a resonator for damping noise in a sound-conducting tubular channel comprising an actively controllable sound generator for generating sound to be superimposed on the tubular channel noise arranged in a resonator chamber, said resonator chamber being connected with the interior of the tubular channel via a sound-transmitting opening in a tube wall of the tubular channel, and a control unit with an input side connected to a sound sensor arranged in the tubular channel, said control unit being connected, as a function of a measurement signal from the sensor containing information about the noise spectrum in the tubular channel, to the sound generator in an amplified way using the same frequency and an inverse phase position; wherein the sound-transmitting opening in the tube wall is a peripheral annular gap in the tube wall.

According to the present invention, the cross-section of the sound-transmitting opening in the tube wall is provided in a ring shape lying essentially perpendicular to a longitudinal axis of the tubular channel, the acoustic waves of the noise actively excited by the sound generator in the resonator chamber penetrating over the entire circumference of the tubular channel and thus, with compact construction of the resonator, being able to efficiently damp the one-dimensional sound wave propagation in the tubular channel. The resonator chamber may be connected to the tubular channel by a plurality of tubular connections or holes positioned in a circumferential arrangemet. The sound-transmitting opening is particularly advantageously constructed as a circumferential annular gap in the tube wall which acts directly on the sound modes in the tubular channel and has a low impedance. In this case, low-frequency modes in particular may also be damped. The annular gap allows the interfering sound to act symmetrically so that the acoustic wave in the tubular channel can be more efficiently influenced and/or extinguished. The dimensioning of the width of the annular gap in the tube wall and the annular gap area are functions of the respective use of the resonator according to the present invention, specifically of the sound spectrum to be damped. Thus, for example, for use in the intake duct of an internal combustion engine, the sound generation and therefore the dimensioning of the gap width and the annular gap area (volume displacement) is particularly also predetermined by the cylinder stroke of the internal combustion engine.

After the sound frequency is detected using a sensor in the tubular channel and the measurement signal of the sensor is relayed to the sound generator by a control unit with antiphase amplification, the noise waves generated in the resonator chamber enter the tubular channel through the annular gap in an especially acoustically active way to generate interference effects in the tubular channel.

### BRIEF DESCRIPTION OF THE DRAWINGS

The invention will be described hereinafter with reference to illustrative preferred embodiments shown in the accompanying drawing figures, in which:

FIG. 1 is a longitudinal sectional view of a sound-conducting tubular channel equipped with a resonator according to the present invention;

FIG. 2 is a circuit diagram of the control electronics of the resonator of FIG. 1;

FIG. 3 shows an alternative resonator design according to the present invention having an annular electric coil as a sound generator,

FIG. 4 shows a variant of the embodiment of FIG. 3 having two annular coils; and

FIG. 5 shows an alternative resonator design according to the present invention having a piezoelement as a sound generator.

### DETAILED DESCRIPTION OF PREFERRED EMBODIMENTS

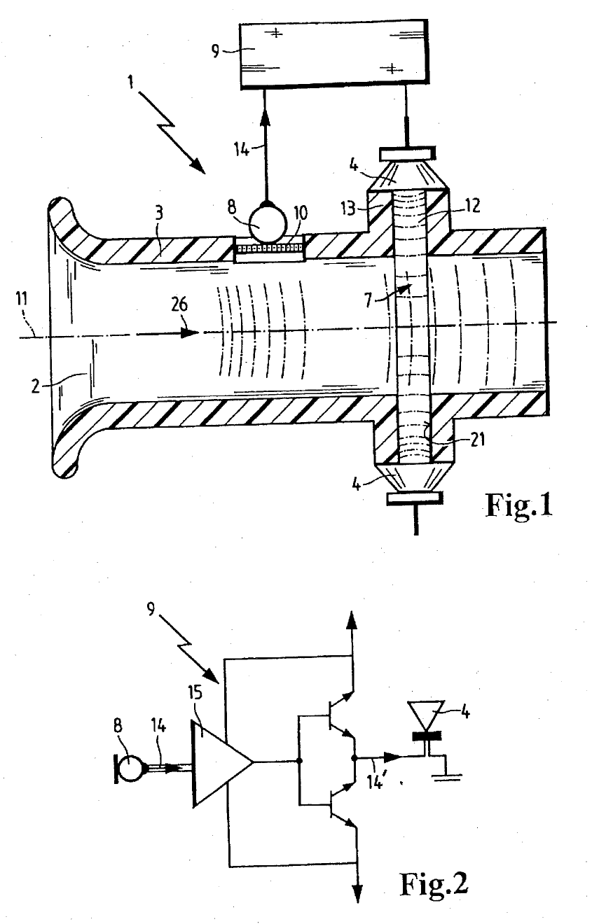

FIG. 1 shows a resonator 1, having a tube wall 3 constructed essentially symmetrically to a longitudinal axis 11. The tubular channel is connected to a gas-conducting pipe system, such as an intake passage of an internal combustion engine, or it may even be a part of the intake pipe. The tubular channel 2 therefore conducts sound which is generated by the internal combustion engine and propagates in the tubular channel 2 in the direction of arrow 26. The resonator 1 for damping the sound in the tubular channel 2 comprises an acoustic sound generator 4, actively controllable by a control unit 9, which generates sound to be superimposed on the noise in the tubular channel. In this case, the measurement signal 14 from a microphone 8 in the tubular channel 2 is input into the control unit 9 and converted by the sound generator 4 into amplified sound having the same frequency but an inverted phase position. The microphone 8 is supported in the tube wall 3 and may be separated by a sound-transmitting seal 10 from the gas-conducting interior of the tubular channel 2.

The optimum measurement position of the microphone would be attained, however, if it lay centrally on the longitudinal axis 11 of the tubular channel 2. In this case, the microphone could be a small tube, supported in the tube wall, which extends to the center of the tubular channel and has an opening for measuring the static sound pressure in the flow-mechanical dead region of the microphone body on the other side of the flow direction 26.

The acoustic sound generator 4 is arranged in a resonator chamber 12, which relays the sound signal emitted by the sound generator 4, having the same frequency as and an inverted phase position relative to the sound in tubular channel 2, through an opening 7 into the tubular channel 2. According to the present invention, the opening 7 is constructed as an annular gap 7, which is oriented in the tube wall 3 at least approximately perpendicular to the longitudinal axis 11 of the tube and is bounded by at least one wall 21, which also extends perpendicular to the longitudinal axis of the tube wall. The annular gap 7 may be a part of the resonator chamber 12 with the acoustic sound generator 4 positioned perpendicular to the longitudinal axis 11 of the tubular channel 2 and thus emitting the sound directly through the resonator chamber 12 and the annular gap 7 for a damping effect on the sound waves, which propagate one-dimensionally in the tubular channel 2.

FIG. 2 is a schematic illustration of the electronics of the control unit 9. The measurement signal 14 from the microphone 8 in the tubular channel 2 (FIG. 1) is connected to an inverting output stage 15 and thus directly drives the acoustic sound generator 4, using an amplified signal 14′ having the same frequency as the measurement signal 14 from the microphone 8, but opposite phase position. For efficient damping, it may be advantageous to integrate suitable band filters and/or delay stages into the amplifier 15.

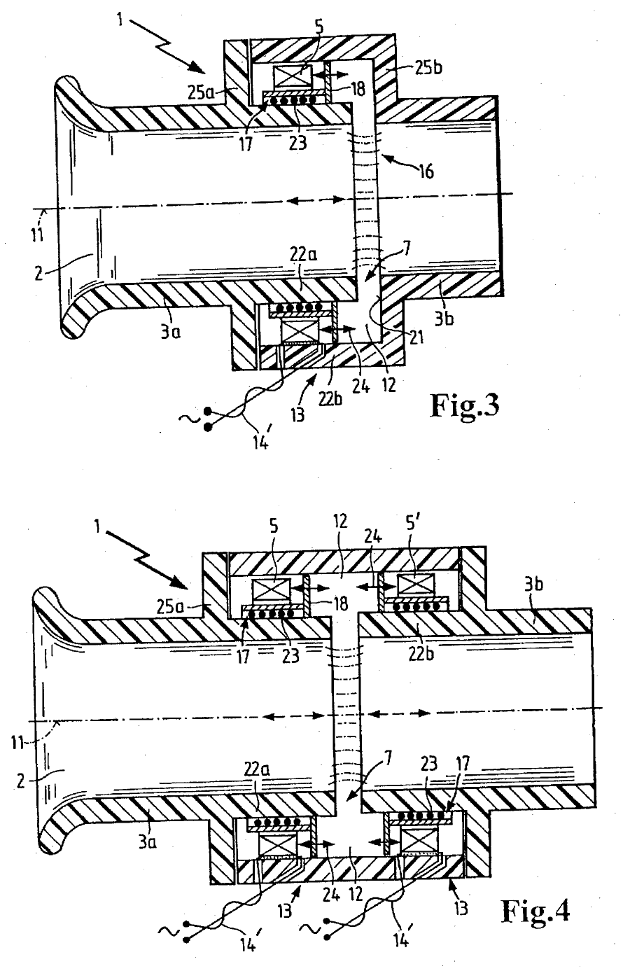

FIG. 3 shows a resonator 1, in which the resonator chamber 12 is configured in an annular shape in a resonator housing 13 surrounding the tubular channel 2. In this case, the resonator housing 13 is formed by overlapping wall sections 22a and 22b, which are the respective end sections of two sectional components 3a and 3b of the tube wall of the tubular channel 2. The resonator chamber 12 is bounded in this case in the axial direction of the tubular channel 2 by radial wall parts of the respective sectional components 3a and 3b. By dividing the resonator housing 13 in accordance with the assembled sectional components 3a and 3b, a very compact construction of the resonator 1 is achieved, the outer wall section of the two overlapping wall sections 22a and 22b being constructed longer than the inner wall section 22a, so that an annular gap 7 remains around the entire circumference of the tube wall of the tubular channel 2.

In the illustrative embodiment of FIG. 3, the sound generator arranged in the resonator chamber 12 comprises an electrically operated annular coil 5 to which the inverted frequency signal 14′ generated by the control unit 9 shown in FIG. 2 is transmitted to generate the noise-cancelling sounds 16. To convert the control signal 14′ into sound waves 16 to be superimposed on the channel noise in the tubular channel 2, a magnetized ferrite tube 17 is provided, which extends axially into the magnetic field of the annular coil 5 in relation to the longitudinal axis 11 of the tubular channel 2. The ferrite tube 17 supports an annular disc 18 or membrane, which forms the boundary of the part of the resonator chamber 12 containing the coil 5 in an essentially airtight manner. The magnetic field is determined by the control signal 14′, and the annular disc 18 follows the ferrite tube 17 in the magnetic field of the coil 5. In this way, sound pressure is generated in the resonator chamber 12 and converted into noise-canceling sound in the annular gap 7 in the tube wall. The ferrite tube is mounted essentially frictionlessly on the outer lateral surface of the sectional component 3a of the tubular channel 2, for example using ball bearings 23 as in the illustrated embodiment.

FIG. 4 shows a variant of the resonator 1, which is constructed similarly to that shown in FIG. 3. In this case, a second actuator in the form of a further coil 5′ is arranged symmetrically relative to the annular gap. Second coil 5′ is operated with the same magnetic field direction in anti-phase or with opposite magnetic field and in phase. Through such an arrangement, the sounds, generated depending on the vibrating mass of the ferrite tube 17 and the membrane and/or annular disc 18, are suppressed, since a mass equalization is produced. The remaining reference numbers for like parts in FIG. 4 correspond to those of FIG. 3.

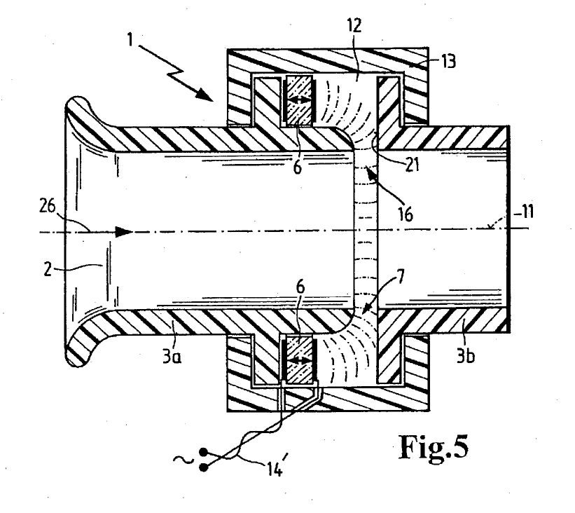

FIG. 5 shows a further embodiment of the resonator according to the present invention, in which the control signal 14′, which contains information about the frequency and phase position of the sound 16 to be generated, is converted into sound 16 by a piezoelectric oscillation element 6 in the resonator chamber 12. The resonator chamber 12 is constructed as an annular cylinder concentric to the longitudinal axis 11 of the tubular channel 2. In this case, in the interest of compact construction of the resonator 1, two adjacent sectional components 3a and 3b of the tube wall of the tubular channel 2 delimit the inner side of the resonator housing 13, while the radially outer side of the resonator housing 13 is formed by a housing part which holds the tube wall parts 3a and 3b together axially. In this illustrative embodiment, the annular gap 7 is adjoined on the side next to the oscillation element 6 by a rounded end of the tube section 3a and on the opposite side by a radial wall 21. In the embodiment of the sound generator as a piezooscillator 6 shown, a higher outlay for circuitry to generate the noise-canceling sound 16 may be necessary depending on the frequency of the noise in the tubular channel 2, and an elevated supply voltage may be necessary in comparison to the embodiments having acoustic sound generators or an annular coil as shown in FIG. 3. However, due to the high resonance frequencies of piezooscillators, a particularly good damping result is achieved at high frequencies from approximately 4 kHz.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A resonator for damping noise in a sound-conducting tubular channel comprising:
an actively controllable sound generator for generating sound to be superimposed on the tubular channel noise arranged in a resonator chamber, said resonator chamber being connected with the interior of the tubular channel via a sound-transmitting opening in a tube wall of the tubular channel, and
a control unit with an input side connected to a sound sensor arranged in the tubular channel, said control unit being connected, as a function of a measurement signal from the sensor containing information about the noise spectrum in the tubular channel, to the sound generator in an amplified way using the same frequency and an inverse phase position;
wherein the sound-transmitting opening in the tube wall is a peripheral annular gap in the tube wall.

2. A resonator according to claim 1, wherein the annular gap is bounded by at least one wall, which extends essentially perpendicular to a longitudinal axis of the tubular channel.

3. A resonator according to claim 1, wherein the sensor is positioned essentially centrally in the tubular channel.

4. A resonator according to claim 3, wherein the sensor is an acoustic microphone positioned, in the direction of the sound in the tubular channel, in front of the annular gap.

5. A resonator according to claim 3, wherein the sensor is mounted in the tube wall.

6. A resonator according to claim 1, wherein the resonator chamber is constructed in the form of a ring around the tubular channel.

7. A resonator according to claim 6, wherein the sound generator is arranged in the resonator chamber perpendicular to the longitudinal axis of the tubular channel.

8. A resonator according to claim 1, wherein the resonator chamber is bounded by a resonator housing in the shape of a ring which surrounds the tubular channel.

9. A resonator according to claim 8, wherein the tubular channel comprises two sectional components with overlapping wall sections which radially bound the resonator housing.

10. A resonator according to claim 1, wherein the sound generator is an electro-acoustic sound generator.

11. A resonator according to claim 1, wherein the sound generator comprises a controllable sound transducer.

12. A resonator according to claim 11, wherein the controllable sound transducer comprises a piezoelectric oscillation element.

13. A resonator according to claim 1, wherein the sound generator includes an active sound transducer element comprising at least one annular electric coil.

14. A resonator according to claim 13, wherein the sound generator comprises two annular coils arranged symmetrically relative to the annular gap.
